(19) United States
(12) Patent Application Publication
(10) Pub. No.: US 2016/0051154 A1
(43) Pub. Date: Feb. 25, 2016

(54) BIOLOGICAL INFORMATION DETECTING DEVICE AND BIOLOGICAL INFORMATION DETECTING METHOD

(71) Applicant: SEIKO EPSON CORPORATION, Tokyo (JP)

(72) Inventor: Takanori IWAWAKI, Hachioji-shi (JP)

(21) Appl. No.: 14/831,769

(22) Filed: Aug. 20, 2015

(30) Foreign Application Priority Data
Aug. 22, 2014 (JP) 2014-169474

(51) Int. Cl. A61B 5/0245; A61B 5/00; A61B 5/11; A61B 5/0205

(52) U.S. Cl. CPC A61B 5/0245; A61B 5/02055; A61B 5/7445; A61B 5/11

(57) ABSTRACT

A biological information detecting device includes a first detection unit detecting biological information of a user, an abnormality determination unit determining whether or not an abnormality occurs in the user on the basis of the biological information detected by the first detection unit, and a frequency change unit changing a detection frequency of the first detection unit to a second frequency higher than a first frequency when the occurrence of the abnormality is determined by the abnormality determination unit.

This application claims priority to Japanese Patent Application No. 2014-169474, filed Aug. 22, 2014, the entirety of which is hereby incorporated by reference.

### BACKGROUND

1. Technical Field

The present invention relates to a biological information detecting device and a biological information detecting method.

2. Related Art

In the related art, a biological information measurement device measuring biological information of a user is known (for example, refer to JP-A-2007-117586).

The biological information measurement device disclosed in JP-A-2007-117586 includes a photoelectric sphygmograph as a measurement unit which senses a subject and stores measurement data, and an analysis unit which performs predetermined data analysis processing with respect to the measurement data acquired by the measurement unit. Among them, the photoelectric sphygmograph is provided with a probe including a sensor unit which measures a photoelectric pulse wave of the subject. In addition, the analysis unit is provided with an operation button group including an abnormal cardiac rhythm analysis button, a defibrillation monitor button, a vascular age analysis button, and an autonomic disturbance analysis button.

In this biological information measurement device, when the abnormal cardiac rhythm analysis button is pressed, photoelectric pulse wave data acquired in advance during a first sampling period (a sampling frequency=30 Hz) is read out, and analysis processing of an abnormal cardiac rhythm is performed on the basis of the photoelectric pulse wave data.

In addition, when the defibrillation monitor button is pressed, the photoelectric pulse wave data is measured in real time in a state where the sampling period is maintained at the first sampling period, and a measurement result is analyzed.

On the other hand, when either the vascular age analysis button or the autonomic disturbance analysis button is pressed, the sampling period is changed to a second sampling period (a sampling frequency=150 Hz) which is comparatively shorter than the first sampling period, and the measured photoelectric pulse wave data is analyzed.

However, in a measurement device such as the photoelectric sphygmograph of the biological information measurement device disclosed in JP-A-2007-117586, the biological information of the photoelectric pulse wave or the like is measured by the device being mounted on the user, and thus when the biological information is measured for a long period of time, a storage capacity for storing the measurement data and a battery capacity for supplying driving power become insufficient. In particular, when the biological information is measured at a comparatively high detection frequency (a sampling frequency) in order to retain and store specific biological information, such a problem becomes remarkable.

For this reason, there has been a demand for a biological information detecting device which is able to detect biological information for a comparatively long period of time and is able to detect specific biological information, as necessary.

### SUMMARY

An advantage of some aspects of the invention is to provide a biological information detecting device which is able to suppress power consumption and is able to specifically detect biological information, and a biological information detecting method.

A first aspect of the invention is directed to a biological information detecting device including a first detection unit detecting biological information of a user, an abnormality determination unit determining whether or not an abnormality occurs in the user on the basis of the biological information detected by the first detection unit, and a frequency change unit changing a detection frequency of the first detection unit to a second frequency higher than a first frequency when the occurrence of the abnormality is determined by the abnormality determination unit.

The change in the detection frequency of the first detection unit to the second frequency may be limited to a time period long enough to acquire a change in the biological information with respect to the occurring abnormality.

According to the first aspect, when the occurrence of the abnormality in the user is determined by the abnormality determination unit on the basis of the biological information detected by the first detection unit operated at the first frequency, the frequency change unit changes the detection frequency of the first detection unit to the second frequency which is higher than the first frequency. According to this, the biological information at the time that the abnormality occurs is able to be accurately detected. At this time, when it is determined that the abnormality occurs, the detection frequency of the first detection unit is changed to the second frequency, and thus it is possible to reduce power consumption and to decrease the storage capacity of the biological information, compared to a case where the biological information is continuously detected at the second frequency from the start of the detection of the biological information detecting device. Accordingly, it is possible to specifically detect the biological information while reducing power consumption.

When the biological information detecting device is a portable detecting device which is used by being mounted on the user, according to the effect described above, it is possible to extend the driving time of the biological information detecting device, compared to a detecting device in which the biological information is continuously detected at the second frequency.

It is preferable that the biological information detecting device according to the first aspect further includes a body motion detection unit detecting body motion of the user, and a body motion determination unit determining whether or not predetermined body motion performed before a timing at which the occurrence of the abnormality is determined by the abnormality determination unit in the past occurs on the basis of a detection result of the body motion detection unit, in which when the occurrence of the predetermined body motion is determined by the body motion determination unit, the frequency change unit changes the detection frequency of the first detection unit to a frequency which is higher than the first frequency and lower than the second frequency.

When the abnormality easily occurs when the user is awoken, body motion at the time that the user is awoken from a resting state including sleeping is able to be exemplified as the predetermined body motion, and when the abnormality easily occurs when the user is at rest, body motion at the time that the user is at rest from an awake state is able to be exemplified as the predetermined body motion.

According to the first aspect with this configuration, when the occurrence of the predetermined body motion performed before the timing at which the occurrence of the abnormality is determined by the abnormality determination unit is determined by the body motion determination unit on the basis of the detection result of the body motion detection unit, the detection frequency of the first detection unit is changed to the frequency which is higher than the first frequency and lower than the second frequency by the frequency change unit. In this case, the detection frequency of the first detection unit is changed to the frequency higher than the first frequency according to the body motion in which the occurrence of the abnormality is predicted, and thus it is possible to accurately determine whether or not the abnormality occurs by the abnormality determination unit on the basis of the detection result of the first detection unit operated at the frequency described above. Accordingly, it is possible to reliably perform specific detection with respect to the biological information at the time that the abnormality occurs.

It is preferable that the biological information detecting device according to the first aspect further includes a counting unit counting a current time, and a time zone determination unit determining whether or not the current time counted by the counting unit enters a time zone including a timing at which the occurrence of the abnormality is determined by the abnormality determination unit in the past, in which when the time zone determination unit determines that the current time counted by the counting unit enters the time zone, the frequency change unit changes the detection frequency of the first detection unit to a frequency which is higher than the first frequency and lower than the second frequency.

When it is determined that the abnormality occurs while the user is awaken, a time zone (for example, a day time zone) in which the user is awake is able to be exemplified as the time zone, and when it is determined that the abnormality occurs while the user is at rest, a time zone (for example, a night time zone) in which the user is at rest is able to be exemplified as the time zone.

According to the first aspect with this configuration, when the entrance of the current time to the time zone including the timing at which the occurrence of the abnormality is determined by the abnormality determination unit in the past, that is, the time zone in which the abnormality easily occurs is determined by the time zone determination unit, the detection frequency of the first detection unit is changed to the frequency which is higher than the first frequency and lower than the second frequency. In this case, according to the time zone in which the occurrence of the abnormality is predicted, the detection frequency of the first detection unit is changed to the frequency higher than the first frequency, and thus it is possible to accurately determine whether or not the abnormality occurs by the abnormality determination unit on the basis of the detection result of the first detection unit operated at the frequency described above. Accordingly, it is possible to reliably perform specific detection with respect to the biological information at the time that the abnormality occurs.

When the biological information detecting device includes each of the body motion determination unit and the time zone determination unit, a change in the detection frequency according to the determination result of the body motion determination unit and a change in the detection frequency according to the determination result of the time zone determination unit maybe independently performed, or may be performed by being combined.

In the latter case, for example, when the current time enters the time zone in which the abnormality easily occurs, the detection frequency of the first detection unit is higher than the first frequency, and when it is determined that the predetermined body motion occurs, the detection frequency may further increase in a range not exceeding the second frequency. According to such a configuration, it is possible to reliably perform specific detection with respect to the biological information at the time that the abnormality occurs.

It is preferable that in the biological information detecting device according to the first aspect, the biological information detected by the first detection unit includes a pulse wave of the user, and the abnormality is an abnormality which is classified into an abnormal cardiac rhythm.

According to the first aspect with this configuration, when the occurrence of the abnormality which is classified as abnormal cardiac rhythm is determined by the abnormality determination unit, the detection frequency of the first detection unit which detects the biological information including the pulse wave is increased from the first frequency to the second frequency. In this case, it is possible to specifically detect the biological information including the pulse wave at the time that the occurrence of the abnormality is determined. Accordingly, it is possible to specifically examine the state of the abnormal cardiac rhythm by analyzing the biological information.

It is preferable that in the biological information detecting device according to the first aspect, the abnormality determination unit determines whether or not the abnormality occurs on the basis of a change in a variation coefficient of a pulse wave interval based on the pulse wave detected by the first detection unit.

Here, when auricular fibrillation which is classified as abnormal cardiac rhythm occurs, the variation coefficient of the pulse wave interval obtained by analyzing the detected pulse wave is considerably changed compared to a normal case.

For this reason, the abnormality determination unit performs determination on the basis of the variation coefficient, and thus it is possible to accurately determine whether or not the auricular fibrillation as the abnormality occurs in the user.

It is preferable that in the biological information detecting device according to the first aspect, at least when a state in which a pulse wave interval based on the pulse wave detected by the first detection unit is shorter than a predetermined first threshold value is continued or when a state in which the pulse wave interval is longer than a second threshold value greater than the first threshold value is continued, the abnormality determination unit determines that the abnormality occurs.

Here, a tachycardia which is classified as abnormal cardiac rhythm is a phenomenon in which a pulse rate (a pulse rate per unit time) increases extremely compared to a normal case, and similarly, a bradycardia which is classified as abnormal cardiac rhythm is a phenomenon in which the pulse rate decreases extremely compared to a normal case. On the other hand, a comparatively short pulse wave interval indicates that the pulse rate is comparatively high, and a comparatively long pulse wave interval indicates that the pulse rate is comparatively low. That is, in a state where the tachycardia occurs, the pulse wave interval is short, and in a state where the bradycardia occurs, the pulse wave interval is long.

For this reason, the abnormality determination unit determines whether a state where the pulse wave interval is shorter than the first threshold value which is an index of the tachycardia is continued or a state where the pulse wave interval is longer than the second threshold value which is greater than the first threshold value and is an index of the bradycardia is continued, and thus it is possible to determine whether or not at least one of the tachycardia and the bradycardia occurs. That is, when the state where the pulse wave interval is shorter than the first threshold value is continued, it is possible to determine that the tachycardia as the abnormality occurs, and when the state where the pulse wave interval is longer than the second threshold value is continued, it is possible to determine that the bradycardia as the abnormality occurs. Accordingly, it is possible to accurately determine whether or not at least one of the tachycardia and the bradycardia occurs in the user.

It is preferable that in the biological information detecting device according to the first aspect, when a waveform of the pulse wave detected by the first detection unit is approximately coincident with a predetermined waveform, the abnormality determination unit determines that the abnormality occurs.

Furthermore, the waveform of a pulse wave at the time that premature contraction occurs or the waveform of a pulse wave at the time that the auricular fibrillation occurs is able to be exemplified as the predetermined waveform.

Here, the premature contraction which is classified as abnormal cardiac rhythm is a phenomenon in which a heart slowly contracts, deviating from an original period. When this premature contraction or the auricular fibrillation described above occurs, the pulse wave shows a unique waveform.

For this reason, the abnormality determination unit determines whether or not the waveform of the pulse wave to be detected is approximately coincident with the predetermined waveform such as the waveform at the time that the premature contraction occurs or the waveform at the time that the auricular fibrillation occurs, and thus it is possible to accurately determine whether or not the premature contraction or the auricular fibrillation occurs.

It is preferable that the biological information detecting device according to the first aspect further includes a frequency setting unit setting the first frequency according to the type of the abnormality.

Here, in a case where the occurrence of the abnormality is determined on the basis of the pulse wave interval and a case where the occurrence of the abnormality is determined on the basis of the waveform of the pulse wave, the detection accuracy of the pulse wave is higher in the latter case than in the former case.

On the other hand, the frequency setting unit sets the first frequency which is the normal detection frequency of the first detection unit according to the type of the abnormality determined by the abnormality determination unit, and thus even when the occurrence of the abnormality is determined on the basis of the waveform of the pulse wave, it is possible to suitably determine the occurrence of the abnormality.

It is preferable that the biological information detecting device according to the first aspect further includes a second detection unit detecting biological information which is different from the biological information detected by the first detection unit, and a detection control unit starting the detection by the second detection unit when the occurrence of the abnormality in the user is determined by the abnormality determination unit.

When the first detection unit detects the pulse wave, an electrocardiograph, a body temperature, and the like are exemplified as the biological information different from the biological information detected by the first detection unit.

According to the first aspect with this configuration, when it is determined that the abnormality occurs, the detection control unit allows the second detection unit to start detection of the other biological information. In this case, a plurality of types of biological information is able to be detected from the time that the abnormality occurs. Accordingly, it is possible to more specifically analyze the status of the abnormality.

A second aspect of the invention is directed to a biological information detecting method performed using a biological information detecting device which detects biological information of a user, the method including: detecting the biological information; determining whether or not an abnormality occurs in the user on the basis of the detected biological information; and increasing a detection frequency of the biological information when it is determined that the abnormality occurs.

By performing the biological information detecting method according to the second aspect using the biological information detecting device, it is possible to obtain the same effects as that of the biological information detecting device according to the first aspect.

### BRIEF DESCRIPTION OF THE DRAWINGS

The invention will be described with reference to the accompanying drawings, wherein like numbers reference like elements.

FIG. 1 is a block diagram illustrating a biological information detection system according to a first embodiment of the invention.

FIG. 2 is a block diagram illustrating a configuration of a control unit of the first embodiment.

FIG. 3 is a diagram illustrating an example of a variation coefficient waveform signal of the first embodiment.

FIG. 4 is a diagram illustrating an example of a pulse wave signal at the time that premature contraction occurs of the first embodiment.

FIG. 5 is a diagram illustrating an example of a RR waveform signal of the first embodiment.

FIG. 6 is a diagram illustrating an example of the RR waveform signal of the first embodiment.

FIG. 7 is a flowchart illustrating biological information detection processing of the first embodiment.

FIG. 8 is a block diagram illustrating a configuration of a control unit of a biological information detecting device configuring a biological information detection system according to a second embodiment of the invention.

FIG. 9 is a flowchart illustrating frequency change processing of the second embodiment.

FIG. 10 is a block diagram illustrating a configuration of a control unit of a biological information detecting device configuring a biological information detection system according to a third embodiment of the invention.

FIG. 11 is a flowchart illustrating frequency change processing of the third embodiment.

### DESCRIPTION OF EXEMPLARY EMBODIMENTS

### First Embodiment

Hereinafter, a first embodiment of the invention will be described with reference to the drawings.

### Configuration of Biological Information Detection System

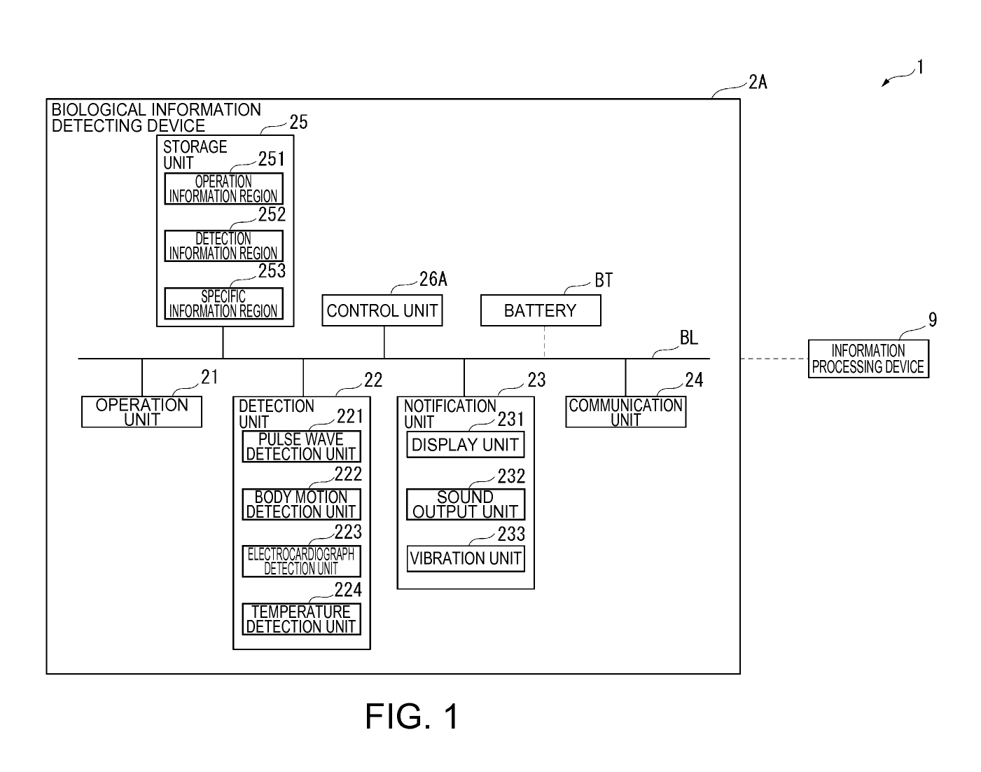

FIG. 1 is a block diagram illustrating a configuration of a biological information detection system 1 according to this embodiment.

As illustrated in FIG. 1, the biological information detection system 1 according to this embodiment includes a biological information detecting device (hereinafter, referred to as a detecting device) 2A and an information processing device 9. In the biological information detection system 1, the detecting device 2A constantly detects biological information and body motion information of a user at a predetermined detection frequency, and detects the biological information at a frequency higher than the predetermined detection frequency when an abnormality (specifically, an abnormality which is classified as an abnormal cardiac rhythm) relevant to the detected biological information occurs.

Among them, the information processing device 9 is connected to the detecting device 2A, accesses a storage unit 25 retained in the detecting device 2A, and acquires and analyzes biological information and operation information stored in the storage unit 25. As the information processing device 9, a Personal Computer (PC) which is able to execute an analysis program of the biological information is able to be exemplified.

### Configuration of Biological Information Detecting Device

Hereinafter, the configuration and the operation of the detecting device 2A will be mainly described.

The detecting device 2A detects the biological information and the body motion information of the user by being mounted on the user, and in this embodiment, detects a pulse wave and an electrocardiograph as the biological information. In addition, the detecting device 2A according to this embodiment detects the body motion of the user. As illustrated in FIG. 1, the detecting device 2A includes an operation unit 21, a detection unit 22, a notification unit 23, a communication unit 24, a storage unit 25, and a control unit 26A which are connected to each other through a bus line BL, and a battery BT which supplies power to these units.

### Configuration of Battery and Operation Unit

In this embodiment, the battery BT is configured of a secondary battery, and is charged by a charging section (not illustrated) using power supplied from the outside.

The operation unit 21 includes a plurality of buttons which is disposed on a case configuring the outer package of the detecting device 2A by being exposed, and outputs an operation signal according to the input (pressed) button to the control unit 26A. Furthermore, the configuration of the operation unit 21 is not limited to the configuration including the button, and may be a configuration including other operation sections such as a touch panel instead of the button or in addition to the button. Further, the operation unit 21 has a configuration which includes an acceleration sensor detecting an acceleration applied to the detecting device 2A, detects a tap operation of the user on the basis of the detected acceleration, and outputs an operation signal according to the tap operation.

### Configuration of Detection Unit

The detection unit 22 detects the biological information and the body motion information of the user under the control of the control unit 26A, and outputs a detection result thereof to the control unit 26A. The detection unit 22 includes a pulse wave detection unit 221, a body motion detection unit 222, an electrocardiograph detection unit 223, and a temperature detection unit 224.

The pulse wave detection unit 221 corresponds to a first detection unit according to the invention, and detects the pulse wave of the user on which the detecting device 2A is mounted. The pulse wave detection unit 221, for example, includes a photoelectric sensor provided with a light emitting element such as a Light Emitting Diode (LED), and a light receiving element such as a photodiode.

The photoelectric sensor detects the pulse wave by emitting light from the light emitting element towards a biological body and by detecting a change in light intensity at the time that the light receiving element receives the light incoming through a blood vessel of the biological body. That is, the light emitted to the biological body is partially absorbed in the blood vessel, the absorptivity in the blood vessel is changed due to an influence of a beat, and thus the light intensity reaching the light receiving element is changed. Then, the control unit 26A described later analyzes a time change (that is, a pulse wave signal) of the light intensity which is detected and output by the light receiving element, and thus, for example, it is possible to acquire a pulse rate (a pulse rate per unit time).

The detection frequency (a sampling rate) of the pulse wave of the photoelectric sensor is set to a normal frequency (a first frequency), for example, 16 Hz in a normal case. However, when specific detection is performed with respect to the pulse wave signal, the detection frequency is changed to a frequency for specific detection (a second frequency), for example, 64 Hz, which is higher than the normal frequency by the control unit 26A. In addition, the normal frequency is also able to be changed by the control unit 26A.

The body motion detection unit 222 detects the body motion information of the user. The body motion detection unit 222 includes the photoelectric sensor for measuring the body motion, and the acceleration sensor.

Among them, the output waveform (a body motion component) of the photoelectric sensor decreases from the output waveform (the pulse wave signal) of the photoelectric sensor for measuring the pulse wave, and thus the detection accuracy of the pulse wave signal is improved.

The acceleration sensor detects an acceleration value according to the operation of the user on which the detecting device 2A is mounted, and outputs an acceleration signal (a body motion signal) indicating the detected acceleration value to the control unit 26A. As the acceleration sensor, a 3-axis sensor which detects the acceleration value on each of an X axis, a Y axis, and a Z axis is able to be exemplified. Furthermore, the acceleration signal which is the detection result of the acceleration sensor is able to be used in processing of reducing noise due to the body motion superimposed on the pulse wave signal detected by the pulse wave detection unit 221.

The electrocardiograph detection unit 223 and the temperature detection unit 224 correspond to a second detection unit according to the invention.

Among them, the electrocardiograph detection unit 223 includes an electrocardiographic sensor which detects the electrocardiograph of the user, and outputs the detection result (an electrocardiographic signal) of the electrocardiographic sensor to the control unit 26A.

In addition, the temperature detection unit 224 includes a temperature sensor which detects the body temperature of the user, and outputs the detection result of the temperature sensor to the control unit 26A.

### Configuration of Notification Unit

The notification unit 23 notifies the user of various information items under the control of the control unit 26A.

The notification unit 23 includes a display unit 231, a sound output unit 232, and a vibration unit 233.

The display unit 231 is configured of various display panels such as a liquid crystal or a plurality of LEDs, and displays the content according to notification information input from the control unit 26A. For example, when the detection frequency is changed in order to perform the specific detection with respect to the biological information such as the pulse wave in detection processing described later, the display unit 231 displays this change, and when the electrocardiograph and the temperature are detected by the electrocardiograph detection unit 223 and the temperature detection unit 224, the display unit 231 displays the detection. Then, the display unit 231 displays the detection result of the detection unit 22.

The sound output unit 232 includes a sound output section such as a speaker, and outputs sound according to sound information input from the control unit 26A.

The vibration unit 233 includes a motor which is controlled by the control unit 26A, and notifies the state of the detecting device 2A using a vibration generated by driving the motor.

### Configuration of Communication Unit

The communication unit 24 includes a communication module which is able to communicate with an external device such as the information processing device 9. The communication unit 24, for example, transmits the information stored in the storage unit 25 to the external device according to a request signal received from the external device. Furthermore, in this embodiment, the communication unit 24 communicates with the external device in a wireless manner, and may communicate with the external device through a relay device such as a cradle, or may communicate with the external device through a cable when the detecting device 2A is connected to the external device through the cable.

### Configuration of Storage Unit

The storage unit 25 is configured of a storage section such as a flash memory, and includes an operation information region 251, a detection information region 252, and a specific information region 253.

The operation information region 251 stores the operation information necessary for operating the detecting device 2A such as various programs and data items. As the operation information, the operation information region 251 stores a control program for controlling the operation of the detecting device 2A, or a biological information detection program for performing the detection processing described later. In addition, as the operation information, the operation information region 251 stores the detection frequency which is able to be set with respect to the detection unit 22 (in particular, the pulse wave detection unit 221).

The detection information region 252 stores various information items detected by the detection unit 22.

The specific information region 253 stores specific biological information detected by the detection processing described later. The specific information region 253 is set in the storage unit 25 separately from the operation information region 251 and the detection information region 252, and thus when the external device (for example, the information processing device 9) which is able to perform the communication through the communication unit 24 accesses the storage unit 25, information stored in the specific information region 253 is easily extracted.

### Configuration of Control Unit

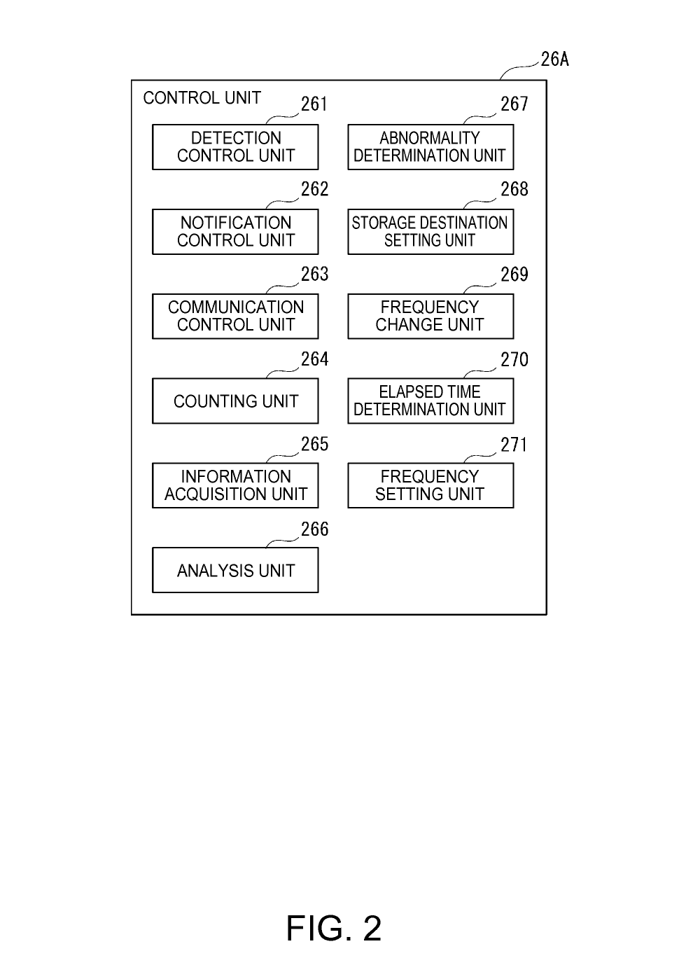

FIG. 2 is a block diagram illustrating the configuration of the control unit 26A.

The control unit 26A includes a control circuit, and controls the operation of the detecting device 2A. The control unit 26A, for example, stores the various information items detected by the detection unit 22 in the storage unit 25, analyzes the pulse wave detected by the pulse wave detection unit 221, and detects the specific biological information by changing the detection frequency of the pulse wave or the like when it is determined that there is an abnormality in the user. As illustrated in FIG. 2, the control unit 26A includes a detection control unit 261, a notification control unit 262, a communication control unit 263, a counting unit 264, an information acquisition unit 265, an analysis unit 266, an abnormality determination unit 267, a storage destination setting unit 268, a frequency change unit 269, an elapsed time determination unit 270, and a frequency setting unit 271, as a function unit which is realized by performing each of the control program and the biological information detection program by the control circuit.

The detection control unit 261 controls the operation of the detection unit 22. Specifically, the detection control unit 261 allows the pulse wave detection unit 221 to detect the pulse wave of the user by at a set detection frequency, and allows the body motion detection unit 222 to detect the acceleration value (the body motion information) at a predetermined detection frequency according to the body motion of the user. In addition, when the occurrence of the abnormality is determined by the abnormality determination unit 267 described later, the detection control unit 261 allows the pulse wave detection unit 221 to detect the pulse wave at the detection frequency which is changed by the frequency change unit 269 in a predetermined time period, and allows the electrocardiograph detection unit 223 and the temperature detection unit 224 to detect the electrocardiograph and the body temperature of the user in the predetermined time period. Furthermore, in this embodiment, the pulse wave detection unit 221 and the body motion detection unit 222 are constantly operated by the detection control unit 261. In addition, the predetermined time period is set to a time including a time period from when the abnormal cardiac rhythm occurs to when the symptomatic state of the abnormal cardiac rhythm subsides, and in this embodiment, is set to 3 minutes as an example.

The notification control unit 262 controls the operation of the notification unit 23. For example, the notification control unit 262 outputs the notification information including display or sound which indicates the operation state of the detecting device 2A, the detection result of the detection unit 22, the changed detection frequency of the pulse wave detection unit 221, and the like to the notification unit 23, and notifies the notification unit of the notification information. In addition, the notification control unit 262, as necessary, drives the motor of the vibration unit 233, and notifies that, for example, the predetermined time period has elapsed by the vibration unit 233.

The communication control unit 263 controls the operation of the communication unit 24 which communicates with the information processing device 9.

The counting unit 264 counts current date and time.

The information acquisition unit 265 performs A/D conversion and amplification with respect to the signal detected by the detection unit 22 which is operated under the control of the detection control unit 261, and acquires detection information including the biological information and the body motion information. Then, the information acquisition unit 265 stores the acquired detection information in the detection information region 252 along with the date and time at which the detection information is detected. In addition, when the occurrence of the abnormality is determined by the abnormality determination unit 267, the information acquisition unit 265 stores the acquired detection information in the specific information region 253 in addition to the detection information region 252.

The analysis unit 266 analyzes the pulse wave signal which is detected by the pulse wave detection unit 221 and acquired by the information acquisition unit 265. Specifically, the analysis unit 266 performs frequency analysis of a predetermined frequency region (for example, a frequency region of 0.25 Hz to 0.5 Hz) of Fast Fourier Transform (FFT) with respect to the acquired pulse wave signal, and calculates a frequency spectrum. Then, the analysis unit 266 generates an RR waveform signal indicating a time change in an RR interval (a time difference between an R wave which is the most acute peak included in the pulse wave signal and an R wave one before the most acute peak) for each frame on the basis of the calculated frequency spectrum. Further, the analysis unit 266 calculates a cardiac beat variation coefficient CVRR in the RR interval, and generates a variation coefficient waveform signal indicating a time change in the cardiac beat variation coefficient CVRR. In addition, the analysis unit 266 counts the number of cardiac beats per unit time on the basis of the acquired pulse wave signal.

The abnormality determination unit 267 determines whether or not the abnormality classified as abnormal cardiac rhythm occurs in the user on the basis of the analysis result of the analysis unit 266. As the abnormality, in this embodiment, it is determined that any one of auricular fibrillation, premature contraction, a tachycardia, and a bradycardia occurs.

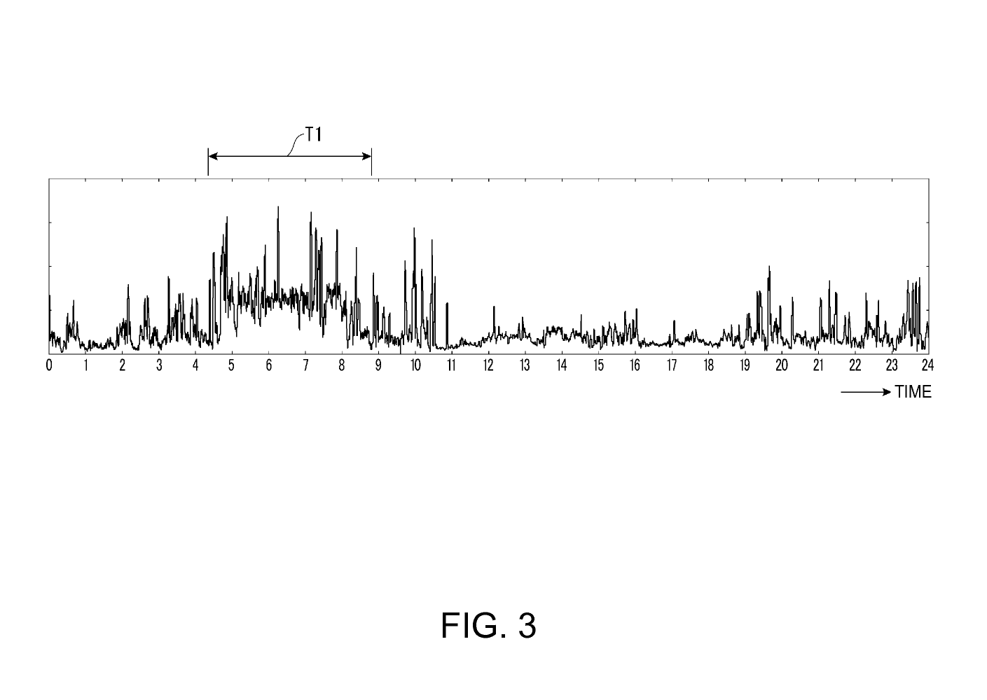

FIG. 3 is a diagram illustrating an example of the variation coefficient waveform signal including a signal at the time that the auricular fibrillation occurs.

Here, the auricular fibrillation is one of the abnormal cardiac rhythms, and indicates a state where the beating rate of an atrium is greater than or equal to 300 times in 1 minute, a heart beats fast and irregularly, and blood stays in the heart. When the auricular fibrillation occurs, the amplitude of the RR waveform signal increases, and as illustrated in a time period T1 of FIG. 3, the cardiac beat variation coefficient CVRR is considerably changed. For this reason, on the basis of this, the abnormality determination unit 267 is able to determine whether or not the auricular fibrillation occurs.

However, the abnormality determination unit 267 is not limited thereto, and the abnormality determination unit 267 may determine whether or not the auricular fibrillation occurs using other methods. For example, the abnormality determination unit 267 may match the waveform of the pulse wave signal at the time that the auricular fibrillation occurs in the past with the waveform of the pulse wave signal acquired by the information acquisition unit 265, and when it is determined that the waveforms are approximately identical to each other, the abnormality determination unit 267 may determine that the auricular fibrillation occurs.

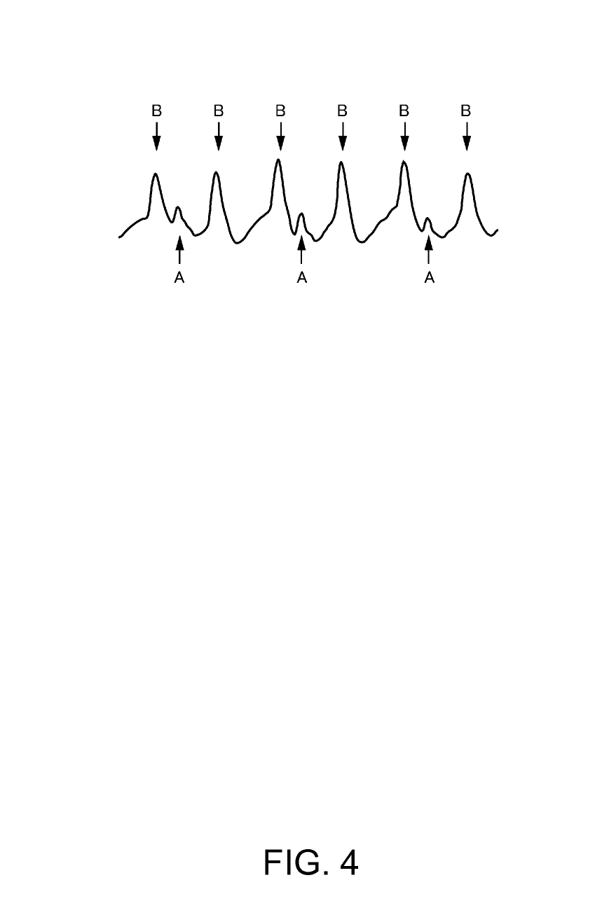

FIG. 4 is a diagram illustrating an example of the pulse wave signal at the time that the premature contraction occurs.

The premature contraction is one of the abnormal cardiac rhythms, and indicates a state where the heart contracts fast, deviating from an original period due to abnormal stimulation. When the premature contraction occurs, as illustrated by an arrow A in FIG. 4, a waveform different from the waveform of the normal sinus rhythm (a waveform indicated by an arrow B) is included in the pulse wave signal. For this reason, the abnormality determination unit 267 matches the waveform at the time that the premature contraction occurs with the waveform of the acquired pulse wave signal, and when it is determined that the waveforms are approximately identical to each other, the abnormality determination unit 267 determines that the premature contraction occurs. Furthermore, the waveform at the time that the premature contraction occurs may be an average waveform, or may be the waveform of the premature contraction which occurred in the user in the past.

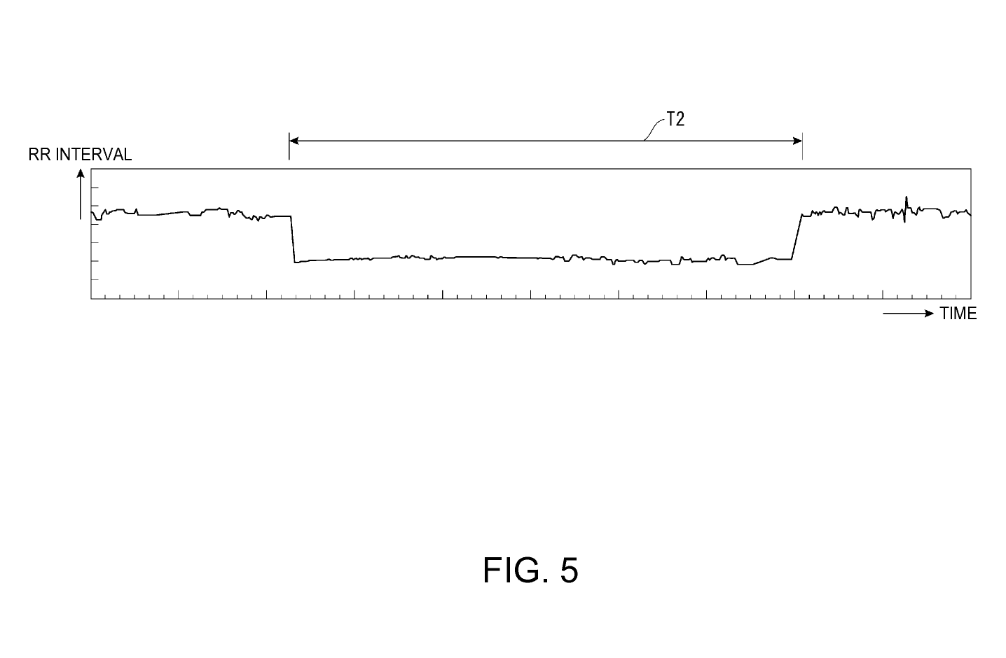

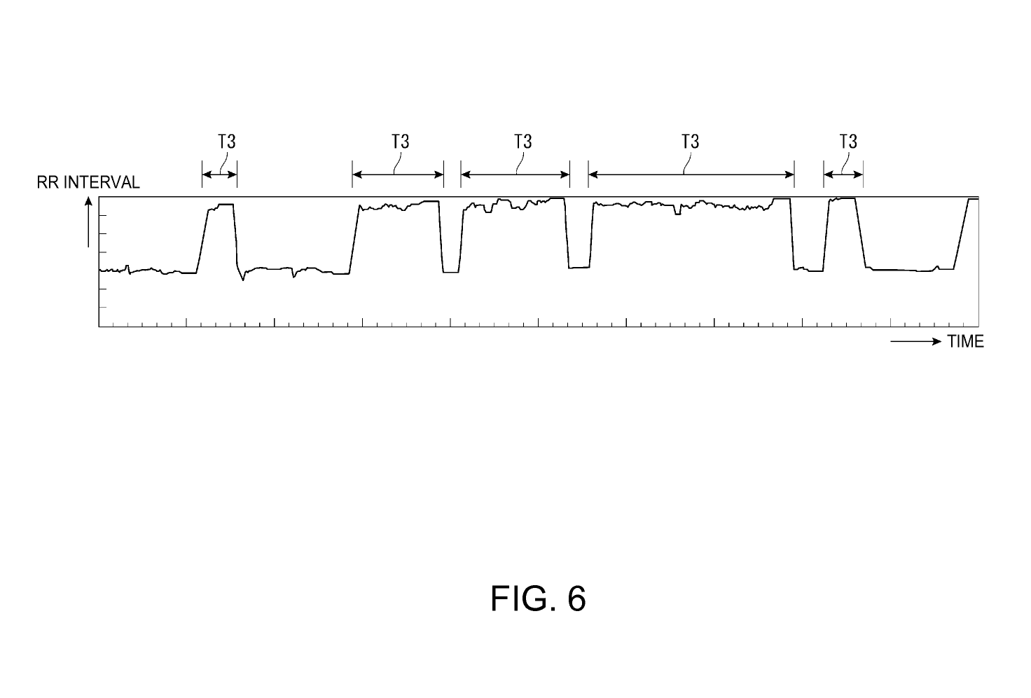

FIG. 5 is a diagram illustrating an example of the RR waveform signal including a signal at the time that the tachycardia occurs. In addition, FIG. 6 is a diagram illustrating an example of the RR waveform signal including a signal at the time that the bradycardia occurs.

The tachycardia indicates a state where a pulse becomes abnormally faster, and the bradycardia indicates a state where the pulse becomes abnormally slower. For example, when the pulse of an average adult of which the number of cardiac beats in a resting state is 60 bpm to 70 bpm is greater than 100 bpm, the tachycardia is assumed, and when the pulse is less than or equal to 50 bpm, the bradycardia is assumed.

Among them, when the tachycardia occurs, as illustrated in a time period T2 of FIG. 5, a state where the RR interval is shorter than that of a normal case is continued, and when the bradycardia occurs, as illustrated in a time period T3 of FIG. 6, a state where the RR interval is longer than that of the normal case is continued. For this reason, the abnormality determination unit 267 determines that the tachycardia occurs when a state where the RR interval is greater than a threshold value of the tachycardia set according to the user is continued for a predetermined period of time, and determines that the bradycardia occurs when a state where the RR interval is less than a threshold value (a threshold value lower than the threshold value of the tachycardia) of the bradycardia set according to the user is continued for a predetermined period of time.

When the abnormality determination unit 267 determines that the abnormality occurs, the storage destination setting unit 268 sets the detection information region 252 and the specific information region 253 as a storage destination of the detection information of the detection unit 22 during the predetermined time period which is set in advance. That is, the detection information is stored in the detection information region 252 by the information acquisition unit 265 as described above when it is not determined that the abnormality occurs, and is stored in not only the detection information region 252 but also the specific information region 253 when it is determined that the abnormality occurs.

When the abnormality determination unit 267 determines that the abnormality occurs, the frequency change unit 269 changes the detection frequency of the pulse wave detection unit 221 to the frequency for specific detection which is higher than the normal frequency during the predetermined time period. In this embodiment, the frequency change unit 269, as described above, changes the normal frequency which is 16 Hz to the frequency for specific detection which is set to 64 Hz during the predetermined time period from a time point when it is determined that the abnormality occurs. Accordingly, the specific pulse wave signal is detected by the pulse wave detection unit 221. In addition, the electrocardiograph detection unit 223 and the temperature detection unit 224 detect the electrocardiograph and the body temperature during the predetermined time period according to the determination of the abnormality determination unit 267 that the abnormality occurs under the control of the detection control unit 261.

The elapsed time determination unit 270 determines whether or not the predetermined time period has elapsed after the abnormality determination unit 267 determines that the abnormality occurs. When the elapsed time determination unit 270 determines that the predetermined time period has elapsed, the frequency change unit 269 returns the detection frequency of the pulse wave detection unit 221 to the frequency before being changed (that is, the normal frequency), and the detection control unit 261 stops the detection of the electrocardiograph detection unit 223 and the temperature detection unit 224. Further, the storage destination setting unit 268 sets the detection information region 252 as the storage destination of the detection information of the detection unit 22 (in this case, the pulse wave signal and the acceleration signal), and awakens the specific information region 253.

The frequency setting unit 271 sets the detection frequency of the pulse wave detection unit 221 in a range not exceeding the frequency for specific detection according to the input operation of the user with respect to the operation unit 21. For example, the determination of the occurrence of the premature contraction is performed by determining whether or not the waveform at the time that the premature contraction occurs is approximately coincident with the waveform of the acquired pulse wave signal. For this reason, when it is necessary to more accurately determine the occurrence of the premature contraction, the frequency setting unit 271, for example, increases the normal frequency to be 32 Hz according to the operation signal input from the operation unit 21 by the input operation of the user.

### Biological Information Detection Processing

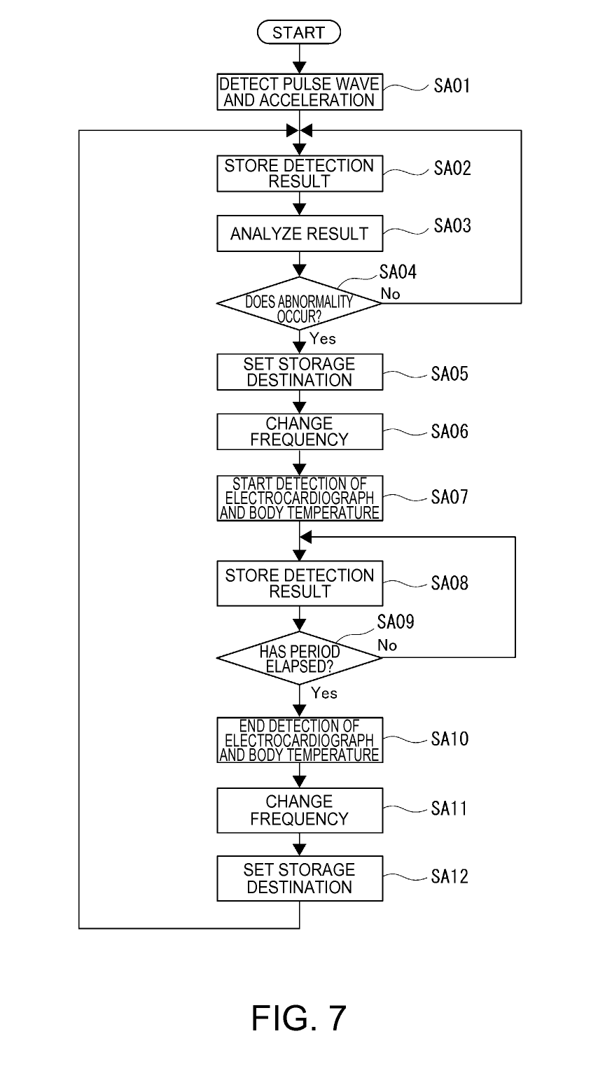

FIG. 7 is a flowchart illustrating the biological information detection processing performed by the detecting device 2A.

The control unit 26A performs the following biological information detection processing according to the detection program stored in the operation information region 251 of the storage unit 25. The biological information detection processing is processing in which the detection frequency of the pulse wave detection unit 221 is changed according to whether or not the abnormality occurs in the user, and when the abnormality occurs, the detection frequency increases, and the pulse wave which is the biological information is specifically detected and recorded.

In the biological information detection processing, as illustrated in FIG. 7, first, the detection control unit 261 allows the pulse wave detection unit 221 and the body motion detection unit 222 to detect the pulse wave and the acceleration (Step SA01). In this case, the detection frequency of the pulse wave detection unit 221 is the normal frequency.

In addition, the information acquisition unit 265 sequentially acquires the detection result of each of the detection units 221 and 222, and stores the detection result in the detection information region 252 of the storage unit (Step SA02).

Then, the analysis unit 266 analyzes the pulse wave signal among the acquired detection results as described above (Step SA03).

After that, the abnormality determination unit 267 determines whether or not the abnormality (at least any one of the auricular fibrillation, the premature contraction, the tachycardia, and the bradycardia) which is classified as abnormal cardiac rhythm occurs in the user on the basis of at least any one of the RR interval which is the analysis result of the analysis unit 266, the RR waveform signal, the variation coefficient waveform signal of the cardiac beat variation coefficient CVRR, and the waveform of the acquired pulse wave signal (Step SA04).

When it is determined that the abnormality does not occur in the determination processing of Step SA04, the biological information detection processing returns to Step SA02, and continuously stores the pulse wave signal and the acceleration signal which are continuously detected in the detection information region 252.

In contrast, when it is determined that the abnormality occurs in the determination processing of Step SA04, the storage destination setting unit 268 adds the specific information region 253 to the storage destination of the detection result of the detection unit 22, and sets the storage destination of the detection result (Step SA05). That is, according to Step SA05, the storage destination of the detection result is the detection information region 252 and the specific information region 253.

In addition, the frequency change unit 269 changes the detection frequency of the pulse wave of the pulse wave detection unit 221 to the frequency for specific detection from the normal frequency (Step SA06).

Further, the detection control unit 261 allows the electrocardiograph detection unit 223 and the temperature detection unit 224 to start the detection of the electrocardiograph and the body temperature of the user (Step SA07).

Furthermore, the sequence of Steps SA05 to SA07 is not limited to the sequence described above, any one of Steps SA05 to SA07 may be processed first, or all of Steps may be processed at the same time.

After Steps SA05 to SA07, the information acquisition unit 265 stores the pulse wave signal, the acceleration signal, the electrocardiograph, and the body temperature which are detected by each of the detection units 221 to 224 in the specific information region 253 along with the detection date and time, and stores the pulse wave signal and the acceleration signal in the detection information region 252 (Step SA08). That is, the pulse wave signal and the acceleration signal are stored in the detection information region 252 as with the normal case, and the pulse wave signal and the acceleration signal, and the electrocardiograph and the body temperature which are newly started to be detected are stored in the specific information region 253.

Then, the elapsed time determination unit 270 determines whether or not the elapsed time after it is determined that the abnormality occurs in the determination processing of Step SA04 exceeds the predetermined time period (Step SA09).

In the determination processing of Step SA09, when it is determined that the elapsed time does not exceed the predetermined time period, the biological information detection processing returns to Step SA08, and continuously stores the various information items which are continuously detected by each of the detection units 221 to 224 in the storage unit 25.

In contrast, in the determination processing of Step SA09, when it is determined that the elapsed time exceeds the predetermined time period, the detection control unit 261 allows the electrocardiograph detection unit 223 and the temperature detection unit 224 to stop the detection of the electrocardiograph and the body temperature (Step SA10).

In addition, the frequency change unit 269 changes the detection frequency of the pulse wave detection unit 221 to the normal frequency from the frequency for specific detection (Step SA11). That is, the detection frequency of the pulse wave detection unit 221 returns to the normal frequency.

Further, the storage destination setting unit 268 sets the detection information region 252 as the storage destination of the detection result of the detection unit 22, and awakens the specific information region 253 (Step SA12).

Then, after Step SA12, the biological information detection processing returns to Step SA02, and thus the information to be detected is only the acceleration according to the pulse wave and the body motion of the user, and the information is stored only in the detection information region 252. Furthermore, as described above, the sequence of Steps SA10 to SA12 is not limited to the sequence described above, any one of Steps SA10 to SA12 may be processed first, or all of Steps may be processed at the same time.

### Effect of First Embodiment

According to the biological information detection system 1 according to this embodiment described above, the following effects are obtained.

When the occurrence of the abnormality in the user is determined by the abnormality determination unit 267 on the basis of the biological information detected by the pulse wave detection unit 221 operated at the first frequency, the frequency change unit 269 changes the detection frequency of the pulse wave detection unit 221 to the frequency for specific detection higher than the normal frequency in the predetermined time period. According to this, it is possible to accurately detect the biological information at the time that the abnormality occurs, it is possible to reduce power consumption compared to a case where the pulse wave is continuously detected at the frequency for specific detection from the start of the detection of the detecting device 2A, and it is possible to decrease the storage capacity of the pulse wave. Further, after the predetermined time period has elapsed, the detection frequency of the pulse wave detection unit 221 is changed to the normal frequency lower than the frequency for specific detection, and thus it is possible to reduce power consumption compared to a case in which the pulse wave is continuously detected at the frequency for specific detection, and it is possible to decrease the storage capacity of the pulse wave. Accordingly, it is possible to specifically detect the pulse wave which is the biological information while reducing the power consumption.

Furthermore, the detecting device 2A is a portable detecting device which is used by being mounted on the user, and thus according to the effects described above, it is possible to extend the driving time of the detecting device 2A compared to a detecting device in which a pulse wave is continuously detected at the frequency for specific detection. For this reason, it is possible to continuously detect the biological information for a comparatively long period of time while mounting the detecting device 2A. Further, by controlling the detection frequency as necessary, it is possible to detect the specific biological information.

Here, the driving time of the detecting device 2A obtained as a time period during which the detecting device 2A is able to continuously detect the biological information is preferably greater than or equal to 4 hours and less than or equal to 24 hours, is more preferably greater than or equal to 1 day and less than or equal to 3 days, and is even more preferably 1 week. When the driving time (a measurement time period) is greater than or equal to 4 hours and less than or equal to 24 hours, the user is able to detect the occurrence of the abnormality in a movement of a time zone or daily life to be paid attention to on the basis of the note received from a medical agency or the like. When the driving time is greater than or equal to 1 day and less than or equal to 3 days, it is possible to grasp a relationship between the daily life rhythm of the user and the occurrence of the abnormality. Further, when the driving time is 1 week, it is possible to grasp a relationship between the lifestyle of the user and the occurrence of the abnormality.

When the occurrence of the abnormality which is classified as abnormal cardiac rhythm is determined by the abnormality determination unit 267, the detection frequency of the pulse wave detection unit 221 detecting the pulse wave increases to the frequency for specific detection from the normal frequency in the predetermined time period. According to this, it is possible to specifically detect the pulse wave at the time that the occurrence of the abnormality is determined. Accordingly, it is possible to specifically examine the state of the abnormal cardiac rhythm by analyzing the detected pulse wave.

When the auricular fibrillation which is classified as abnormal cardiac rhythm occurs, the variation coefficient of the pulse interval is considerably changed compared to a normal case. For this reason, the abnormality determination unit 267 performs the determination on the basis of the variation coefficient, and thus it is possible to accurately determine whether or not the auricular fibrillation occurs in the user.

The abnormality determination unit 267 determines whether or not a state where the pulse interval based on the detected pulse wave is shorter than the threshold value which is the index of the tachycardia is continued for a predetermined period of time and whether or not a state where the pulse is greater than the threshold value which is the index of the tachycardia and is longer than the threshold value which is the index of the bradycardia is continued for a predetermined period of time, and thus it is possible to determine whether or not the tachycardia and the bradycardia occur. That is, when the state where the pulse interval is shorter than the threshold value of the tachycardia is continued for a predetermined period of time, it is possible to determine that the tachycardia occurs, and when the state where the pulse interval is longer than the threshold value of the bradycardia is continued for a predetermined period of time, it is possible to determine that the bradycardia occurs. Accordingly, it is possible to accurately determine whether or not the tachycardia and the bradycardia occur in the user.

The abnormality determination unit 267 determines whether or not the waveform of the detected pulse wave is approximately coincident with a predetermined waveform such as the waveform at the time that the premature contraction occurs or the waveform at the time that the auricular fibrillation occurs. According to this, the abnormality determination unit 267 is able to accurately determine whether or not the premature contraction or the auricular fibrillation occurs in the user.

Here, in a case where the occurrence of the abnormality is determined on the basis of the pulse interval and in a case where the occurrence of the abnormality is determined on the basis of the waveform of the pulse wave, the detection accuracy of the pulse wave, in other words, the sampling frequency of the pulse wave is higher in the latter case than in the former case. Then, the occurrence of the premature contraction is determined by whether or not the waveform of the detected pulse wave signal is approximately coincident with the waveform of the pulse wave signal at the time that the premature contraction occurs.

On the other hand, the frequency setting unit 271 is able to set the normal frequency of the pulse wave detection unit 221 according to the type of the abnormality which is determined by the abnormality determination unit 267, and thus even the occurrence of the abnormality is determined on the basis of the waveform of the pulse wave signal, it is possible to suitably determine the occurrence of the abnormality. For this reason, for example, the type of the abnormality to be monitored is specified on the basis of a clinical examination result in the medical agency, and the frequency setting unit 271 may set the normal frequency of the pulse wave detection unit 221 on the basis of the type of the specified abnormality. In this case, the user may operate the operation unit 21 and input the numerical value of the normal frequency, and the frequency setting unit 271 may set the normal frequency described above to the frequency of the input numerical value. Alternatively, the user may select the type of the abnormality to be detected, and the frequency setting unit 271 may set the normal frequency described above to the frequency corresponding to the type of the selected abnormality.

When the occurrence of the abnormality is determined by the abnormality determination unit 267, the detection control unit 261 allows the electrocardiograph detection unit 223 to detect the electrocardiographic waveform and the temperature detection unit 224 to detect the body temperature. According to this, it is possible to detect a plurality of types of biological information from the time that the abnormality occurs. Accordingly, it is possible to specifically analyze the status of the abnormality.

### Second Embodiment

Next, a second embodiment of the invention will be described.

A biological information detection system according to this embodiment has the same configuration as that of the biological information detection system 1 described above. However, the biological information detection system is different from the biological information detection system 1 described above in that when a predetermined body motion in which the occurrence of the abnormality is predicted is performed, the detection frequency of the pulse wave detection unit 221 is higher than the normal frequency, and when the abnormality occurs, the detection frequency becomes higher. Furthermore, in the following description, the same reference numerals are applied to the same parts or the approximately same parts as those described above, and the description thereof will be omitted.

### Configuration of Biological Information Detection System

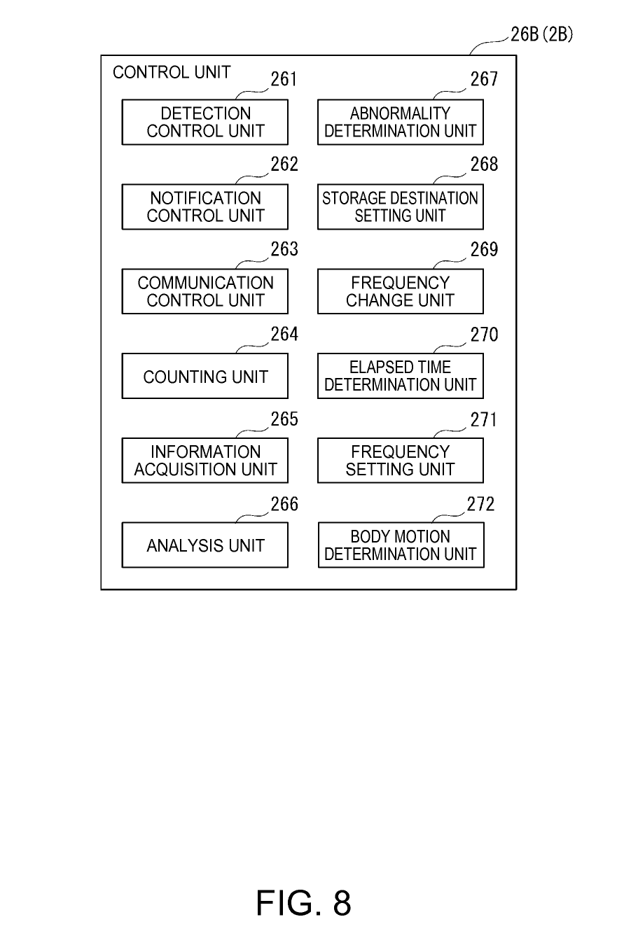

FIG. 8 is a block diagram illustrating a configuration of a control unit 26B provided in a biological information detecting device 2B of the biological information detection system according to this embodiment.

The biological information detection system according to this embodiment has the same configuration and the same function as that of the biological information detection system 1 described above except that the biological information detecting device 2B is provided instead of the biological information detecting device 2A. In addition, the detecting device 2B has the same configuration and the same function as that of the detecting device 2A described above except that the control unit 26B illustrated in FIG. 8 is provided instead of the control unit 26A.

Here, as a body in which the abnormality (in particular, the auricular fibrillation) classified as abnormal cardiac rhythm occurs, a body in which the abnormality easily occurs in an awake state where a sympathetic nerve predominates, that is, a sympathetic nerve type (a day type) body, and a body in which the abnormality easily occurs in a resting state such as sleeping where a parasympathetic nerve predominates, that is, a parasympathetic nerve type (a night type) body are included.

On the other hand, in the detecting device 2B according to this embodiment, the occurrence timing of the abnormality is acquired on the basis of the pulse wave signal acquired in a detection time period (a time period of greater than or equal to 1 day) in the past, and the user determines whether the body is the sympathetic nerve type body or the parasympathetic nerve type body on the basis of a change in the acceleration signal acquired at a timing other than the occurrence timing (for example, the acceleration signal acquired in a predetermined time period other than the occurrence timing). Furthermore, the predetermined time period is a time period during which it is possible to determine whether the user is in the resting state or in the awake state at the abnormality occurring timing on the basis of the acquired acceleration signal, and as the predetermined time period, for example, 30 minutes is able to be exemplified.

Then, in a case where the user is the sympathetic nerve type body, when it is determined that the user is in the awaken state on the basis of the acceleration signal to be detected, the frequency change unit 269 changes the detection frequency of the pulse wave detection unit 221 to a frequency for prediction which is higher than the normal frequency, and when it is determined that the abnormality occurs, the detection frequency further increases to the frequency for specific detection in the predetermined time period. In this case, for example, when it is determined that the user is in the resting state such as sleeping on the basis of the acceleration signal, the detection frequency of the pulse wave detection unit 221 is set to the normal frequency of 16 Hz, when it is determined that the user is in the awaken state where the occurrence of the abnormality is predicted, the detection frequency increases to the frequency for prediction of 32 Hz, and when it is determined that the abnormality occurs, the detection frequency is set to the frequency for specific detection of 64 Hz in the predetermined time period.

In contrast, in a case where the user is the parasympathetic nerve type body, when there is no change in the acceleration signal, and it is determined that the user is in the resting state, the frequency change unit 269 changes the detection frequency of the pulse wave detection unit 221 to the frequency for prediction higher than the normal frequency, and when it is determined that the abnormality occurs, the detection frequency further increases to the frequency for specific detection in the predetermined time period. In this case, for example, when it is determined that the user is in the awaken state on the basis of the acceleration signal, the detection frequency of the pulse wave detection unit 221 is set to the normal frequency of 16 Hz, when it is determined that the user is in the resting state (including a sleep state, a relaxed state of the awake state, and the like), the detection frequency is set to the frequency for prediction of 32 Hz, and when it is determined that the abnormality occurs, the detection frequency is set to the frequency for specific detection of 64 Hz in the predetermined time period.

The detection frequency is set in this way, and thus as illustrated in FIG. 8, the control unit 26B includes a body motion determination unit 272 which determines whether the basis of the user is in the awaken state or in the resting state on the basis of the acceleration signal which is changed according to the body motion, in addition to the function units 261 to 271 described above.

Specifically, the body motion determination unit 272 functions after the occurrence of the abnormality is determined by the abnormality determination unit 267 at least once. The body motion determination unit 272 determines whether the abnormality occurrence timing is a timing at which the user is in the awaken state or a timing at which the user is in the resting state on the basis of the acceleration signal until the timing at which the occurrence of the abnormality is determined by the abnormality determination unit 267 in the past. In other words, the body motion determination unit 272 determines whether the user is the sympathetic nerve type body or the parasympathetic nerve type body. Then, the body motion determination unit 272 determines whether or not an abnormality prediction body motion which is a body motion at the time the current time enters a prediction time period in which the occurrence of the abnormality is predicted occurs, and determines whether or not a time period termination body motion which is a body motion indicating that the prediction time period terminates occurs, on the basis of the acceleration signal input from the body motion detection unit 222.

That is, when it is determined that the user is the sympathetic nerve type body, the body motion determination unit 272 sets a body motion at the time that the state of the user is moved from the resting state to the awaken state as the abnormality prediction body motion, and when the change in the acceleration signal to be detected is approximately coincident with a change in the acceleration signal at the time that the abnormality prediction body motion occurs, the body motion determination unit 272 determines that the abnormality prediction body motion occurs. In addition, in this case, when the change in the acceleration signal to be detected is approximately coincident with a change in the acceleration signal at the time that the state of the user is moved from the awaken state to the resting state, the body motion determination unit 272 determines that the time period termination body motion occurs.

In contrast, when it is determined that the user is the parasympathetic nerve type body, the body motion determination unit 272 sets a body motion at the time that the state of the user is moved from the awaken state to the resting state as the abnormality prediction body motion, and when the change in the acceleration signal to be detected is approximately coincident with the change in the acceleration signal at the time that the abnormality prediction body motion occurs, the body motion determination unit 272 determines that the abnormality prediction body motion occurs. In addition, in this case, when the change in the acceleration signal to be detected is approximately coincident with a change in the acceleration signal at the time that the state of the user is moved from the resting state to the awake state, the body motion determination unit 272 determines that the time period termination body motion occurs.

Furthermore, at the time of determining whether or not the abnormality prediction body motion occurs and whether or not the time period termination body motion occurs, the body motion determination unit 272 may perform the determination on the basis of not only the acceleration signal acquired from the body motion detection unit 222 but also the pulse wave signal acquired from the pulse wave detection unit 221, and may perform the determination on the basis of the pulse rate calculated from the pulse wave signal.

Thus, when the occurrence of the abnormality prediction body motion is determined by the body motion determination unit 272, the frequency change unit 269 changes the detection frequency of the pulse wave detection unit 221 to a detection frequency for abnormality prediction which is higher than the normal frequency and lower than the frequency for specific detection.

In addition, when the occurrence of the time period termination body motion is determined by the body motion determination unit 272, the frequency change unit 269 changes the detection frequency of the pulse wave detection unit 221 to the normal frequency.

### Frequency Change Processing

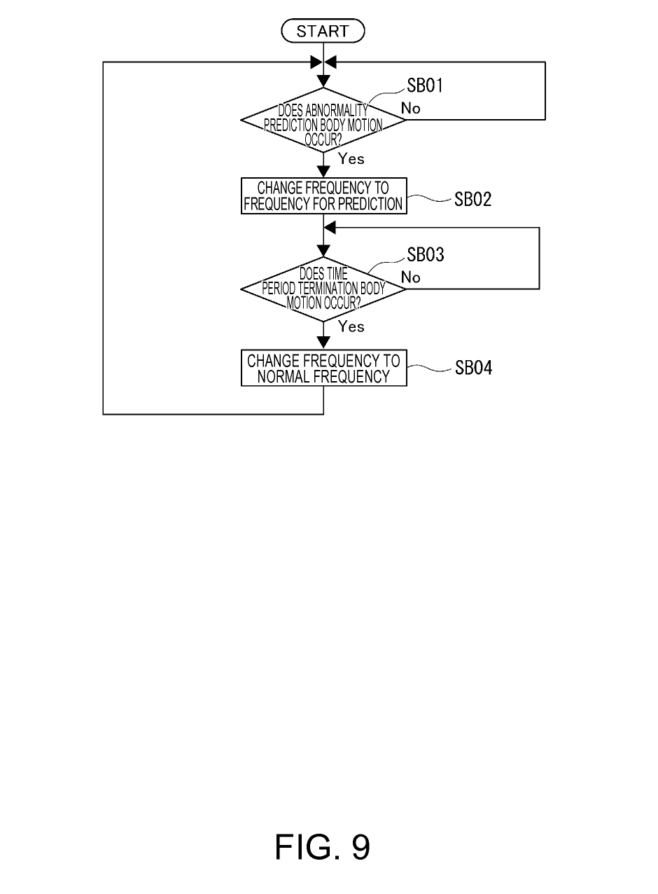

FIG. 9 is a flowchart illustrating frequency change processing.

The detecting device 2B performs the following frequency change processing along with the biological information detection processing performed by the detecting device 2A in parallel. The frequency change processing is processing which is performed according to a frequency change program included in the biological information detection program described above, and as described above, is performed at least once after the occurrence of the abnormality is determined by the abnormality determination unit 267.

In the frequency change processing, as illustrated in FIG. 9, first, the body motion determination unit 272 determines whether or not the abnormality prediction body motion occurs (Step SB01).

Here, when it is determined that the abnormality prediction body motion does not occur, the frequency change processing returns to Step SB01, and the determination processing of Step SB01 is repeatedly performed at predetermined intervals.

In contrast, when it is determined that the abnormality prediction body motion occurs, the frequency change unit 269 changes the detection frequency of the pulse wave detection unit 221 to the frequency for prediction from the normal frequency (Step SB02). At this time, when the detection frequency of the pulse wave detection unit 221 is changed to the frequency for specific detection in the biological information detection processing, the frequency change unit 269 changes the detection frequency of the pulse wave detection unit 221 to the frequency for prediction after the predetermined time period has elapsed.

After Step SB02, the body motion determination unit 272 determines whether or not the time period termination body motion occurs (Step SB03).

Here, when it is determined that the time period termination body motion does not occur, the frequency change processing returns to Step SB03, and the determination processing of Step SB03 is repeatedly performed at predetermined intervals.

In contrast, when it is determined that the time period termination body motion occurs, the frequency change unit 269 changes the detection frequency of the pulse wave detection unit 221 to the normal frequency from the frequency for prediction (Step SB04). At this time, when the detection frequency of the pulse wave detection unit 221 is changed to the frequency for specific detection in the biological information detection processing, as described above, the frequency change unit 269 changes the detection frequency of the pulse wave detection unit 221 to the normal frequency after the predetermined time period has elapsed.

After Step SB04, the frequency change processing returns to Step SB01, and the frequency change processing is repeatedly performed.

### Effect of Second Embodiment

According to the biological information detection system according to this embodiment described above, the same effects as those of the biological information detection system 1 described above are able to be obtained, and the following effects are also able to be obtained.

When the body motion determination unit 272 determines that the abnormality prediction body motion which is approximately identical to the body motion performed before the timing at which the occurrence of the abnormality is determined by the abnormality determination unit 267 in the past occurs on the basis of the detection result of the body motion detection unit 222, the frequency change unit 269 changes the detection frequency of the pulse wave detection unit 221 to the frequency for prediction which is higher than the normal frequency and lower than the frequency for specific detection. According to this, the detection frequency is changed to the frequency for prediction higher than the normal frequency according to the body motion in which the occurrence of the abnormality is predicted, and thus it is possible to accurately determine whether or not the abnormality occurs on the basis of the detection result of the pulse wave detection unit. Accordingly, it is possible to reliably perform the specific detection with respect to the biological information at the time that the abnormality occurs.

### Third Embodiment

Next, a third embodiment of the invention will be described.

A biological information detection system according to this embodiment as the same has the same configuration as that of the biological information detection system described above. Here, in the biological information detection system of the second embodiment, the detection frequency of the pulse wave detection unit 221 becomes higher than that of the normal case according to the occurrence of the abnormality prediction body motion, and thus the determination accuracy of the abnormality occurrence is improved. On the other hand, in a detecting device including the biological information detection system according to this embodiment, the detection frequency becomes higher than that of the normal casein a time zone in which the occurrence of the abnormality is predicted. In this point, the biological information detection system according to this embodiment is different from the biological information detection system described above. Furthermore, in the following description, the same reference numerals are applied to the same parts or the approximately same parts as those described above, and the description thereof will be omitted.

### Configuration of Biological Information Detection System

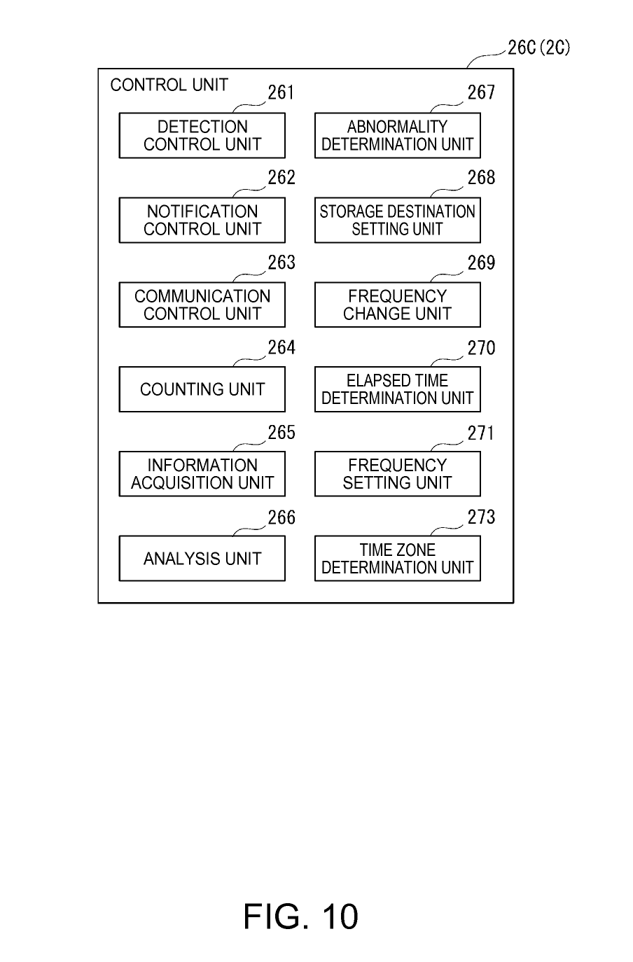

FIG. 10 is a block diagram illustrating a configuration of a control unit 26C provided in a biological information detecting device 2C of the biological information detection system according to this embodiment.

The biological information detection system according to this embodiment has the same configuration and the same function as that of the biological information detection system of the second embodiment described above except that the biological information detecting device 2C is provided instead of the biological information detecting device 2B. In addition, the detecting device 2C has the same configuration and the same function as that of the detecting device 2B described above except that the control unit 26C illustrated in FIG. 10 is provided instead of the control unit 26B.

The control unit 26C has the same configuration and the same function as that of the control unit 26B described above except that a time zone determination unit 273 is provided instead of the body motion determination unit 272.

The time zone determination unit 273 functions after the occurrence of the abnormality is determined by the abnormality determination unit 267 at least once. The time zone determination unit 273 determines whether it is day time or night time on the basis of a time counted by the counting unit 264 at the time that the occurrence of the abnormality is determined by the abnormality determination unit 267. In other words, time zone determination unit 273 determines whether the user is the sympathetic nerve type body in which the abnormality occurs in the awake state or the parasympathetic nerve type body in which the abnormality occurs in the resting state (including sleeping). Then, the time zone determination unit 273 determines whether or not the time of the current date and time counted by the counting unit 264 is included in a time zone in which the abnormality easily occurs.

For example, when the time of determining the occurrence of the abnormality by the abnormality determination unit 267 in the past is day time (for example, 6 a.m. to 6 p.m.), the time zone determination unit 273 sets an abnormality prediction time zone to 6 a.m. to 6 p.m., and determines whether or not the current time enters the abnormality prediction time zone and whether or not the current time is deviated from the abnormality prediction time zone.

In contrast, when the time of determining the occurrence of the abnormality by the abnormality determination unit 267 is night time (for example, 6 p.m. to 6 a.m. of the next day), the time zone determination unit 273 set the abnormality prediction time zone to 6 p.m. to 6 a.m. of the next day, and determines whether or not the current time enters the abnormality prediction time zone and whether or not the current time is deviated from the abnormality prediction time zone.

When the time zone determination unit 273 determines that the current time enters the abnormality prediction time zone set in this way, the frequency change unit 269 changes the detection frequency of the pulse wave detection unit 221 to the frequency for prediction (for example, 32 Hz) which is higher than the normal frequency (for example, 16 Hz) and is lower than the frequency for specific detection (for example, 64 Hz).

In addition, when the time zone determination unit 273 determines that the current time is deviated from the abnormality prediction time zone, the frequency change unit 269 changes the detection frequency of the pulse wave detection unit 221 to the normal frequency. Furthermore, when the control unit 26C determines that the battery voltage of the battery BT is lower than a predetermined threshold value, the frequency change unit 269 sets the detection frequency of the pulse wave detection unit 221 to the normal frequency even when the current time enters the abnormality prediction time zone.

### Frequency Change Processing

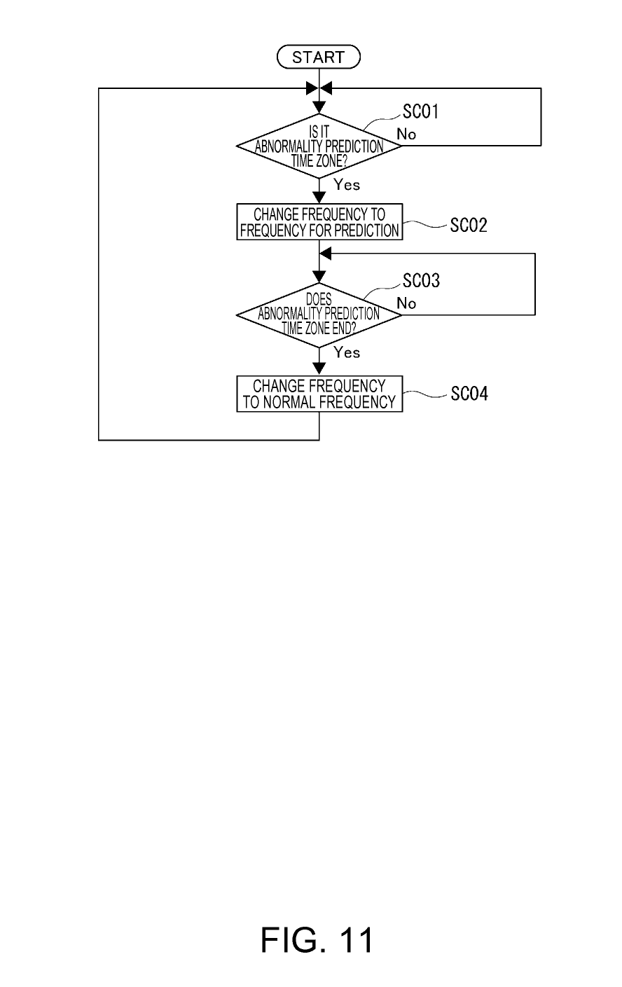

FIG. 11 is a flowchart illustrating frequency change processing.

The detecting device 2C independently performs the following frequency change processing along with the biological information detection processing performed by the detecting device 2A. The frequency change processing is processing performed according to the frequency change program included in the biological information detection program described above, and as described above, is performed at least once after the occurrence of the abnormality is determined by the abnormality determination unit 267.

In the frequency change processing, as illustrated in FIG. 11, first, the time zone determination unit 273 determines whether or not the current time counted by the counting unit 264 enters the abnormality prediction time zone (Step SC01).

In the determination processing, when it is determined that the current time does not enter the abnormality prediction time zone, the frequency change processing returns to Step SC01, and the determination processing of Step SC01 is repeatedly performed at predetermined intervals.

In contrast, when it is determined that the current time enters the abnormality prediction time zone, the frequency change unit 269 changes the detection frequency of the pulse wave detection unit 221 to frequency for prediction from the normal frequency (Step SC02). At this time, when the detection frequency of the pulse wave detection unit 221 is changed to the frequency for specific detection in the biological information detection processing described above, the frequency change unit 269 changes the detection frequency to the frequency for prediction after the predetermined time period has elapsed.

After Step SC02, the time zone determination unit 273 determines whether or not the current time is deviated from the abnormality prediction time zone (Step SC03).

In the determination processing, when it is determined that the current time is not deviated from the abnormality prediction time zone, the frequency change processing returns to Step SC03, and the determination processing of Step SC03 is repeatedly performed at predetermined intervals.

In contrast, when it is determined that the current time is deviated from the abnormality prediction time zone, the frequency change unit 269 changes the detection frequency of the pulse wave detection unit 221 to the normal frequency from the frequency for prediction (Step SC04). At this time, when the detection frequency of the pulse wave detection unit 221 is changed to the frequency for specific detection in the biological information detection processing described above, as described above, the frequency change unit 269 changes the detection frequency to the normal frequency after the predetermined time period has elapsed.

After Step SC04, the frequency change processing returns to Step SC01, and the frequency change processing is repeatedly performed.

### Effect of Third Embodiment

According to the biological information detection system according to this embodiment described above, the same effects as those of the biological information detection system 1 described above are able to be obtained, and the following effects are also able to be obtained.

When the time zone determination unit 273 determines that the current time is in the predetermined time zone including the timing at which the occurrence of the abnormality is determined by the abnormality determination unit 267 in the past, that is, the time zone in which the abnormality easily occurs, the detection frequency of the pulse wave detection unit 221 is changed to the frequency which is higher than the normal frequency and lower than the frequency for specific detection. According to this, the detection frequency becomes higher than the normal frequency according to the time zone in which the occurrence of the abnormality is predicted, and thus it is possible to accurately determine whether or not the abnormality occurs on the basis of the detection result of the pulse wave detection unit 221 operated at the detection frequency. Accordingly, it is possible to reliably perform the specific detection with respect to the biological information at the time that the abnormality occurs.

Furthermore, the control unit 26C of this embodiment does not include the body motion determination unit 272. However, the control unit 26C may also include the body motion determination unit 272. In this case, the frequency change processing of the frequency change unit 269 according to the determination result of the body motion determination unit 272 and the frequency change processing according to the determination result of the time zone determination unit 273 may be independently performed, or these frequency change processings may be combined.

In the latter case, for example, when the time zone determination unit 273 determines that the current time enters the abnormality prediction time zone, the frequency change unit 269 changes the detection frequency of the pulse wave detection unit 221 to a first frequency for prediction (for example, 24 Hz) which is higher than the normal frequency and lower than the frequency for specific detection. Then, when the body motion determination unit 272 determines that the abnormality prediction body motion occurs while the current time is included in the abnormality prediction time zone, the detection frequency may be changed to a second frequency for prediction (for example, 32 Hz) which is higher than the first frequency for prediction and lower than the frequency for specific detection. Further, in addition to this change processing of the detection frequency or instead of the change processing, when it is determined that the current time enters the abnormality prediction time zone, the detection frequency of the body motion detection unit 222 may become higher than that of the normal case, and when it is determined that the current time is deviated from the abnormality prediction time zone, the detection frequency of the body motion detection unit 222 may return to that of the normal case.

### Modification of Embodiment

The invention is not limited to each of the embodiments, and modifications, improvements, and the like within a range where the object of the invention is able to be attained are included in the invention.

In each of the embodiments, the predetermined time period in which the detection frequency is changed to the frequency for specific detection corresponding to the second frequency is 3 minutes. However, the invention is not limited thereto, the predetermined time period is able to be suitably changed insofar as a change in the pulse wave signal relevant to the abnormally is able to be detected.

In each of the embodiments, the normal frequency corresponding to the first frequency is set to 16 Hz, and the frequency for specific detection corresponding to the second frequency is set to 64 Hz. In addition, in the second embodiment and the third embodiment, the frequency for prediction is set to 32 Hz, and in the third embodiment, the first frequency for prediction is set to 24 Hz and the second frequency for prediction is set to 32 Hz. However, the invention is not limited thereto, and the value of each frequency is able to be suitably changed. That is, in order of an increasing value, the normal frequency, the frequency for prediction (the first frequency for prediction and the second frequency for prediction), and the frequency for specific detection are arranged, and the second frequency for prediction may be higher than the first frequency for prediction.

In addition, when the abnormality prediction body motion occurs, the detection frequency may be changed to the frequency for specific detection from the normal frequency.

In each of the embodiments, the pulse wave detection unit 221 as the first detection unit detects the pulse wave as the biological information. However, the invention is not limited thereto. For example, as the biological information, the other biological information such as a cardiac beat may be detected. In addition, the abnormality of which the occurrence is determined by the abnormality determination unit 267 is not limited to the abnormally classified as abnormal cardiac rhythm, and may be the other abnormality insofar as the abnormality is an abnormality relevant to the biological information which is able to be detected by the detection unit 22, in other words, an abnormality of which the occurrence is able to be determined by the detected biological information.

In each of the embodiments, the abnormality determination unit 267 determines the occurrence of the auricular fibrillation, the premature contraction, the tachycardia, and the bradycardia which are respectively classified as abnormal cardiac rhythm using the method described above. However, the invention is not limited thereto. That is, the occurrence of the abnormality may be determined using the other method.

In addition, the abnormality of which the occurrence is determined by the abnormality determination unit 267 is not limited to the auricular fibrillation, the premature contraction, the tachycardia, and the bradycardia, and in addition to these or instead of at least one among them, the occurrence of the other abnormal cardiac rhythm such as atrial flutter may be determined.

In each of the embodiments, the frequency setting unit 271 sets the value of the normal frequency according to the input operation of the user with respect to the operation unit 21. However, the invention is not limited thereto, and the frequency setting unit 271 may not be included. In contrast, the frequency setting unit 271 may set not only the value of the normal frequency but also the value of each of the frequency for specific detection and the frequency for prediction (the first frequency for prediction and the second frequency for prediction).

In each of the embodiments, the detection unit 22 includes the electrocardiograph detection unit 223 and the temperature detection unit 224. However, the invention is not limited thereto, and the electrocardiograph detection unit 223 and the temperature detection unit 224 may not be included, and in this case, Step SA07 described above may be omitted. In contrast, the electrocardiograph detection unit 223 and the temperature detection unit 224 may constantly detect the electrocardiographic detect and the temperature detect. In addition, the detection unit 22 may include a detection unit detecting the other biological information.

In addition, in the first embodiment and the third embodiment, the detection unit 22 includes the body motion detection unit 222. However, the invention is not limited thereto, and in the detecting devices 2A and 2C of the first embodiment and the third embodiment, the body motion detection unit 222 may not be included.

In each of the embodiments, when the occurrence of the abnormality is determined by the abnormality determination unit 267, the storage destination setting unit 268 adds the specific information region 253 which is easily read from the outside as the storage destination of the detection result of the detection unit 22, and the information acquisition unit 265 stores the detection result in the detection information region 252 and the specific information region 253. However, the invention is not limited thereto. For example, the detection result of the detection unit 22 may be stored only in the detection information region 252, and the storage destination setting unit 268 and the specific information region 253 may not be included. In addition, when it is determined that the abnormality occurs, the detection result maybe stored in the detection information region 252, and when it is determined that the abnormality does not occur, the detection result may not be stored.

In addition, the storage unit 25 may not be configured of one store device, and each of the operation information region 251, the detection information region 252, and the specific information region 253 may be configured of a different store device.

In each of the embodiments, the notification unit 23 includes the display unit 231, the sound output unit 232, and the vibration unit 233. However, the invention is not limited thereto. For example, the notification unit 23 may not be included, and even when the notification unit 23 is included, at least any one of the display unit 231, the sound output unit 232, and the vibration unit 233 may not be included.

In the second embodiment described above, when it is determined that the abnormality prediction body motion occurs, the frequency change unit 269 changes the detection frequency of the pulse wave detection unit 221 to the frequency for prediction which is higher than the normal frequency and lower than the frequency for specific detection. The abnormality prediction body motion is not limited to the example described above, and may be other body motions. For example, when characteristic body motion is detected in the predetermined time period until the abnormality occurrence timing, the body motion may be set as the abnormality prediction body motion. As the characteristic body motion, for example, a movement of greater than or equal to a predetermined strength, a movement of greater than or equal to a predetermined pulse rate, a standing-up operation from a sitting position or a horizontal position, a body condition based on an autonomic nerve activity state obtained by analyzing the pulse wave, bath, driving a vehicle or the like, maintaining the same position for a long period of time, and the like are included.

In addition, when it is determined that the time period termination body motion occurs, the frequency change unit 269 changes the detection frequency of the pulse wave detection unit 221 to the normal frequency. However, the configuration is not limited thereto, and the frequency change unit 269 may change the detection frequency to the normal frequency after the predetermined time period has elapsed in which the detection frequency is changed to the frequency for specific detection according to the occurrence of the abnormality.

In the third embodiment described above, when it is determined that the current time enters the abnormality prediction time zone, the detection frequency of the pulse wave detection unit 221 is changed to the frequency for prediction which is higher than the normal frequency and lower than the frequency for specific detection, and when it is determined that the current time is deviated from the abnormality prediction time zone, the detection frequency is changed to the normal frequency. The abnormality prediction time zone is not limited to the time zone of 6 a.m. to 6 p.m. or the time zone of 6 p.m. to 6 a.m. of the next day, and is able to be suitably changed. For example, when the abnormality is likely to occur in the user at the time of sleeping, the abnormality prediction time zone may be set to a time zone of 10 p.m. to 7 a.m. of the next day in conformity to the bed time and the rising time of the user. In addition, for example, when the abnormality is likely to occur in the user in the awake state, the abnormality prediction time zone may be set to a time zone of 6 a.m. to 10 p.m. in conformity to the rising time and the bed time of the user. The time is able to be suitably changed. Further, the abnormality prediction time zone may be set according to the input operation of the user which is performed with respect to the operation unit 21.

Further, the time zone is not limited to the day time and night time, and for example, 1 day may be divided into 3 or more time zone by dividing the time zone every 8 hours. In addition, each time zone is not limited to a time zone of the same hours, and as described above, at least one of a plurality of time zones may be shorter than others.

In addition, the predetermined time period including the time at which the occurrence of the abnormality is determined in the past may be set to the abnormality prediction time zone. In addition, the control unit 26C may confirm the battery voltage of the battery BT, and may set the time period of the abnormality prediction time zone on the basis of the battery voltage. For example, the time period of the abnormality prediction time zone may be set on the basis of the battery voltage such that when the battery voltage is greater than or equal to 70% of the maximum value of the battery BT (when it is determined that the battery capacity of the battery BT is greater than or equal to 70% on the basis of the battery voltage), the time period of the abnormality prediction time zone is set to 8 hours, when it is determined that the battery voltage is greater than or equal to 40% and less than 70% of the maximum value of the battery BT, the time period is set to 4 hours, and when it is determined that the battery voltage is less than 40% of the maximum value of the battery BT, the time period is set to 2 hours.

In each of the embodiments, the detection unit 22 includes the pulse wave detection unit 221, the body motion detection unit 222, the electrocardiograph detection unit 223, and the temperature detection unit 224. On the other hand, a position detection unit detecting the current location of the user may be included. As the position detection unit, a configuration including a receiver which corresponds to a satellite positioning system such as a Global Positioning System (GPS), GLONASS, GALILEO, and quasi-zenith, and calculates and outputs position information from a satellite signal received from a position information satellite, and a device which calculates the position information using radio waves for communication is able to be exemplified.

When such a position detection unit is included, the control unit 26 may store the current position of the user at the time that the abnormality occurs as an abnormality occurrence location in association with the occurrence time of the abnormality or the detected biological information. In this case, when it is determined that the user visits again the occurrence location or is in the vicinity of the location on the basis of the position information detected by the position detection unit, the frequency setting unit 271 may change the detection frequency to the frequency for specific detection or the frequency for prediction from the normal frequency. Accordingly, it is possible to analyze the occurrence of the abnormality from a viewpoint of the location, and thus it is possible to control the pulse wave detection unit 221 while reflecting the taste, the life pattern, and the movement pattern of the user.

In the description above, the embodiment is divided according to the characteristic and is described as the first embodiment, the second embodiment, and the third embodiment for the understanding of the invention, and the processing of the embodiments may be performed in cooperation. For example, the biological information detected by each of the detection units 221, 223, and 224, the body motion information based on the acceleration signal detected by the body motion detection unit 222, and the position information detected by the position detection unit are stored in association with the date and time information indicating the date and time at which the occurrence of the abnormality is determined, and when the occurrence of the abnormality is detected or presage of the abnormality is detected on the basis of at least any one of the biological information, the state information, the position information, and the date and time information, the detection frequency may be changed to the frequency for specific detection or the frequency for prediction from the normal frequency.

What is claimed is:

1. A biological information detecting device, comprising:
a first detection unit detecting biological information of a user;
an abnormality determination unit determining whether or not an abnormality occurs in the user on the basis of the biological information detected by the first detection unit; and
a frequency change unit changing a detection frequency of the first detection unit to a second frequency higher than a first frequency when the occurrence of the abnormality is determined by the abnormality determination unit.

2. The biological information detecting device according to claim 1, further comprising:
a body motion detection unit detecting body motion of the user; and
a body motion determination unit determining whether or not predetermined body motion performed before a timing at which the occurrence of the abnormality is determined by the abnormality determination unit in the past occurs on the basis of a detection result of the body motion detection unit,
wherein when the occurrence of the predetermined body motion is determined by the body motion determination unit, the frequency change unit changes the detection frequency of the first detection unit to a frequency which is higher than the first frequency and lower than the second frequency.

3. The biological information detecting device according to claim 1, further comprising:
a counting unit counting a current time; and
a time zone determination unit determining whether or not the current time counted by the counting unit enters a time zone including a timing at which the occurrence of the abnormality is determined by the abnormality determination unit in the past,
wherein when the time zone determination unit determines that the current time counted by the counting unit enters the time zone, the frequency change unit changes the detection frequency of the first detection unit to a frequency which is higher than the first frequency and lower than the second frequency.

4. The biological information detecting device according to claim 1,
wherein the biological information detected by the first detection unit includes a pulse wave of the user, and
the abnormality is an abnormality which is classified as an abnormal cardiac rhythm.

5. The biological information detecting device according to claim 4,
wherein the abnormality determination unit determines whether or not the abnormality occurs on the basis of a change in a variation coefficient of a pulse wave interval based on the pulse wave detected by the first detection unit.

6. The biological information detecting device according to claim 4,
wherein at least when a state in which a pulse wave interval based on the pulse wave detected by the first detection unit is shorter than a predetermined first threshold value is continued or when a state in which the pulse wave interval is longer than a second threshold value greater than the first threshold value is continued, the abnormality determination unit determines that the abnormality occurs.

7. The biological information detecting device according to claim 4,
wherein when a waveform of the pulse wave detected by the first detection unit is approximately coincident with a predetermined waveform, the abnormality determination unit determines that the abnormality occurs.

8. The biological information detecting device according to claim 1, further comprising:
a frequency setting unit setting the first frequency according to the type of the abnormality.

9. The biological information detecting device according to claim 1, further comprising:
a second detection unit detecting biological information which is different from the biological information detected by the first detection unit; and
a detection control unit starting the detection by the second detection unit when the occurrence of the abnormality in the user is determined by the abnormality determination unit.

10. A biological information detecting method performed using a biological information detecting device which detects biological information of a user, the method comprising:
detecting the biological information;
determining whether or not an abnormality occurs in the user on the basis of the detected biological information; and
increasing a detection frequency of the biological information when it is determined that the abnormality occurs.
